(19) United States
(12) Patent Application Publication
(10) Pub. No.: US 2019/0247405 A1
(43) Pub. Date: Aug. 15, 2019

(54) TREATMENT OF SMA

(71) Applicant: Hoffmann-La Roche Inc., Little Falls, NJ (US)

(72) Inventors: Thomas Blaettler, Basel (CH); Kathleen Dorothy McCarthy, Basel (CH); Patricia Sanwald Ducray, Basel (CH); Franziska Schaedeli Stark, Basel (CH)

(73) Assignee: Hoffmann-La Roche Inc., Little Falls, NJ (US)

(21) Appl. No.: 16/201,877

(22) Filed: Nov. 27, 2018

Related U.S. Application Data
(63) Continuation of application No. PCT/EP2017/063101, filed on May 31, 2017.

(30) Foreign Application Priority Data
Jun. 3, 2016 (EP) 16172972.8

(51) Int. Cl. A61K 31/575; A61K 9/00; A61P 25/28

(52) U.S. Cl. CPC A61K 31/575; A61P 25/28; A61K 9/0053

(57) ABSTRACT

The present invention relates to olesoxime for use in the treatment of spinal muscular atrophy (SMA), its pharmaceutical composition to be used in the treatment of SMA, its methods of treatment thereof.

The invention relates to olesoxime for use in the treatment of spinal muscular atrophy (SMA), its pharmaceutical composition to be used in the treatment of SMA, its methods of treatment thereof.

Spinal muscular atrophy (SMA), in its broadest sense, describes a collection of inherited and acquired central nervous system (CNS) diseases characterized by progressive motor neuron loss in the spinal cord and brainstem causing muscle weakness and muscle atrophy. SMA is characterized by a degeneration of the alpha motor neurons from the anterior horn of the spinal cord leading to muscular atrophy and resulting in paralysis. This alpha motor neuron degeneration thus substantially compromises the vital prognosis of patients. In healthy subjects, these neurons transmit messages from the brain to the muscles, leading to the contraction of the latter. In the absence of such a stimulation, the muscles atrophy. Subsequently, in addition to a generalized weakness and atrophy of the muscles, and more particularly of those of the trunk, upper arms and thighs, these disorders can be accompanied by serious respiratory problems.

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. The clinical spectrum of SMA disorders has been divided into the following five groups:
1) Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.
2) Type I SMA (Infantile SMA or Werdnig-Hoffmann disease) presents symptoms between 0 and 6 months, form of SMA is also very severe. Patients never achieve the ability to sit, and death usually occurs within the first 2 years without ventilatory support.
3) Type II SMA (Intermediate SMA) has an age of onset at 7-18 months. Patients achieve the ability to sit unsupported, but never stand or walk unaided. Prognosis in this group is largely dependent on the degree of respiratory involvement.
4) Type III SMA (Juvenile SMA or Kugelberg-Welander disease) is generally diagnosed after 18 months. Type 3 SMA individuals are able to walk independently at some point during their disease course but often become wheelchair-bound during youth or adulthood.
5) Type IV SMA (Adult onset SMA). Weakness usually begins in late adolescence in the tongue, hands, or feet, then progresses to other areas of the body. The course of adult SMA is much slower and has little or no impact on life expectancy.

All the forms of spinal muscular atrophy are accompanied by progressive muscle weakness and atrophy subsequent to the degeneration of the neurons from the anterior horn of the spinal cord. SMA currently constitutes one of the most common causes of infant mortality. It equally affects girls or boys in all regions of the world with a prevalence of between 1/6000 and 1/10 000.

Currently an effective treatment does not exist to check the neuronal degenerations. A therapeutic approach for protecting the neurons from dying is the supply of neurotrophic proteins.

Olesoxime has been investigated as cytoprotective agents in clinical phase II. The supposed mechanism of action of Olesoxime is linked to the protection of the SMN neurons-deficient motor neurons from neurodegeneration.

A randomized double blind, placebo-controlled, phase 2 study on olesoxime (NTC01302600) was performed on SMA type II and type III patients aged 3 to 25 years. The trial showed that olesoxime 100 mg/ml oral liquid suspension formulation at a dose of 10 mg/kg per day was well tolerated and demonstrated potential to maintain motor function. The outcome of the study seems to suggest that additional investigations may be beneficial for the development of olesoxime.

Surprisingly upon analysis of the data, it was identified that improvement in motor function would be possible by increasing the dose significantly to a dose above 15 mg/kg per day, in particular between 15 mg/kg to 40 mg/kg per day. In particular the most effect is expected to be observed at a dose between 20 mg/kg per day to 30 mg/kg per day.

Additional studies wherein the protocols are described herein below in the example section will be started to confirm this surprising finding.

### BRIEF DESCRIPTION OF THE FIGURES

FIG. 1: Predicted Exposure-Effect Relationship at week 104 (Population mean prediction with 95% CI)

FIG. 2: Olesoxime at 30 mg/kg/day s.c Improves Survival of Transgenic NSE-Cre; SMNF7/F7 Mice.

All publications, patent applications, patents and other references mentioned herein are incorporated by reference in their entirety.

The nomenclature used in the present application is based on IPUAC systematic nomenclature, unless indicated otherwise.

Various features and embodiments of the present invention are disclosed herein, however other features of the invention, modifications and equivalents will be apparent to a person skilled in the relevant art, based on the teachings provided. The invention described is not limited to the examples and embodiments provided, various alternatives equivalents will be appreciate by those skilled in the art. As used herein, the singular forms “a”, “an” and “the” include the plural unless the context clearly dictates otherwise. For example, “a” individual will also include “individuals”.

Unless otherwise stated, the following terms used in the specification and claims have the meanings given below:

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In a particular embodiment of the invention the subject is a human with spinal muscular atrophy (SMA). In another specific embodiment, the subject is a human with SMA caused by an inactivating mutation or deletion in the SMN1 gene on both chromosomes, resulting in a loss of SMN1 gene function.

The term “spinal muscular atrophy” (or SMA) relates to a disease caused by an inactivating mutation or deletion in the SMN1 gene on both chromosomes, resulting in a loss of SMN1 gene function. Symptoms of SMA include muscle weakness, poor muscle tone, weak cry, weak cough, limpness or a tendency to flop, difficulty sucking or swallowing, difficulty breathing, accumulation of secretions in the lungs or throat, clenched fists with sweaty hand, flickering/vibrating of the tongue, head often tilted to one side, even when lying down, legs that tend to be weaker than the arms, legs frequently assuming a “frog legs” position, feeding difficulties, increased susceptibility to respiratory tract infections, bowel/bladder weakness, lower-than-normal weight, inability to sit without support, failure to walk, failure to crawl, and hypotonia, areflexia, and multiple congenital contractures (arthrogryposis) associated with loss of anterior horn cells.

The term “treating spinal muscular atrophy (SMA)” or “treatment of spinal muscular atrophy (SMA)” includes one or more of the following effects: (i) reduction or amelioration of the severity of SMA; (ii) delay of the onset of SMA; (iii) inhibition of the progression of SMA; (iv) reduction of hospitalization of a subject; (v) reduction of hospitalization length for a subject; (vi) increase of the survival of a subject; (vii) improvement of the quality of life of a subject; (viii) reduction of the number of symptoms associated with SMA; (ix) reduction of or amelioration of the severity of one or more symptoms associated with SMA; (x) reduction of the duration of a symptom associated with SMA; (xi) prevention of the recurrence of a symptom associated with SMA; (xii) inhibition of the development or onset of a symptom of SMA; and/or (xiii) inhibition of the progression of a symptom associated with SMA. More particular, “treating SMA” denotes one or more of the following beneficial effects: (i) a reduction in the loss of muscle strength; (ii) an increase in muscle strength; (iii) a reduction in muscle atrophy; (iv) a reduction in the loss of motor function; (v) an increase in motor neurons; (vii) a reduction in the loss of motor neurons; (viii) protection of SMN deficient motor neurons from degeneration; (ix) an increase in motor function; (x) an increase in pulmonary function; and/or (xi) a reduction in the loss of pulmonary function.

In detail, “treating SMA” results in the functional ability or helps retain the functional ability for a human infant or a human toddler to sit up unaided or for a human infant, a human toddler, a human child or a human adult to stand up unaided, to walk unaided, to run unaided, to breathe unaided, to turn during sleep unaided, or to swallow unaided.

The term “mg/kg” refers to the dose in milligram of olesoxime being used per kilogram of body weight of the mammal to be treated, in particular a human (i. e. a male or female human). For example, 20 mg/kg means a dose of 20 milligram of olesoxime per kilogram of body weight of the mammal to be treated, in particular a human (i. e. a male or female human).

The term “mg/ml” refers to the amount of olesoxime in milligram per volume of oil in milliliter.

The term “at more than 15 mg/kg” as used herein may in particular refer to “at dose of more than 15 mg/kg” or “at a dosage of more than 15 mg/kg”.

The term “at 15 mg/kg to 40 mg/kg” as used herein may in particular refer to “at dose of 15 mg/kg to 40 mg/kg” or “at a dosage of 15 mg/kg to 40 mg/kg”.

The term “at 15 mg/kg to 30 mg/kg” as used herein may in particular refer to “at dose of 15 mg/kg to 30 mg/kg” or “at a dosage of 15 mg/kg to 30 mg/kg”.

The term “at 20 mg/kg to 30 mg/kg” as used herein may in particular refer to “at dose of 20 mg/kg to 30 mg/kg” or “at a dosage of 20 mg/kg to 30 mg/kg”.

The term “at 20 mg/kg” as used herein may in particular refer to “at a dose of 20 mg/kg” or “at a dosage of 20 mg/kg”.

The term “active pharmaceutical ingredient” (or “API”) denotes the compound or molecule in a pharmaceutical composition that has a particular biological activity.

The term “pharmaceutical composition” refers to a preparation which is in such form as to permit the biological activity of an active ingredient contained therein to be effective, and which contains no additional components which are unacceptably toxic to a subject to which the composition would be administered. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

Olesoxime according to the present invention refers to a compound of formula (I)

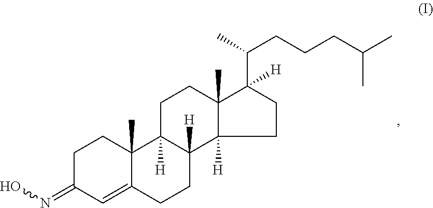

a mixture of compounds of formula (I′) and formula (I″)

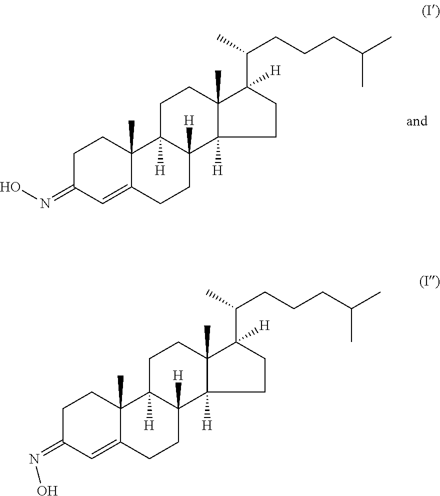

also known as TRO19622, CAS Number 22033-87-0, Cholest-4-en-3-one oxime, [(8S,9S,10R,13R,14S,17R)-10,13-dimethyl-17-[(2R)-6-methylheptan-2-yl]-1,2,6,7,8,9,11,12,14,15,16,17-dodecahydrocyclopenta[a]phenanthren-3-ylidene]hydroxylamine or a mixture (NZ)—N-[(8S,9S,10R,13R,14S,17R)-10,13-dimethyl-17-[(2R)-6-methylheptan-2-yl]-1,2,6,7,8,9,11,12,14,15,16,17-dodecahydrocyclopenta[a]phenanthren-3-ylidene]hydroxylamine and (NE)-N-[(8S,9S,10R,13R,14S,17R)-10,13-dimethyl-17-[(2R)-6-methylheptan-2-yl]-1,2,6,7,8,9,11,12,14,15,16,17-dodecahydrocyclopenta[a]phenanthren-3-ylidene]hydroxylamine. Methods of making and using the compound are described in EP1601363 A1.

Methods of making and using the pharmaceutical composition is described in EP2124961 A1.

In one embodiment, the invention provides olesoxime for use in the treatment of SMA at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a particular embodiment, the invention provides olesoxime for use in the treatment of SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA or/and type III SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA or type III SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA and type III SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type III SMA in mammal (in particular a mammal in need thereof), particularly wherein the mammal is a human (such as a male or female human), at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

Most particularly, olesoxime for use in the treatment of SMA, particularly type II SMA or/and type III SMA, at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In another embodiment, the invention provides olesoxime for use in the treatment of SMA, wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a particular embodiment, the invention provides olesoxime for use in the treatment of SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA or/and type III SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA or type III SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA and type III SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type II SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a more particular embodiment, the invention provides olesoxime for use in the treatment of type III SMA in mammal (in particular a mammal in need thereof), particularly wherein the mammal is a human (such as a male or female human), wherein olesoxime is being administered, in particular administered orally at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg. More particularly, olesoxime for use in the treatment of SMA, particularly type II SMA or/and type III SMA, at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In one embodiment, the invention provides a method for the treatment of spinal muscular atrophy (SMA), comprising administering to a mammal (in particular a mammal in need thereof), particularly wherein the mammal is a human (such as a male or female human), olesoxime at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg. Particularly, the invention provides a method for the treatment of spinal muscular atrophy (SMA), comprising administering to a mammal (in particular a mammal in need thereof), particularly wherein the mammal is a human (such as a male or female human), by oral administration olesoxime at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In a particular embodiment, the invention provides a method for the treatment of spinal muscular atrophy (SMA), comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of spinal muscular atrophy (SMA), comprising administering to a mammal (in particular a mammal in need thereof) particularly wherein the mammal is a human (such as a male or female human), by oral administration olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. Most particularly the invention provides a method for the treatment of SMA, wherein olesoxime is administer once a day, in particular with food.

In a further particular embodiment, the invention provides a method for the treatment of type II SMA or/and type III SMA, comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of type II SMA or/and type III SMA, comprising administering to a mammal (in particular a mammal in need thereof), particularly wherein the mammal is a human (such as a male or female human), by oral administration olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day.

In an another particular embodiment, the invention provides a method for the treatment of type II SMA or type III SMA, comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of type II SMA or type III SMA, comprising administering to a mammal (in particular a mammal in need thereof), particularly wherein the mammal is a human (such as a male or female human), by oral administration olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day.

In an another particular embodiment, the invention provides a method for the treatment of type II SMA and type III SMA, comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of type II SMA and type III SMA, comprising administering to a mammal (in particular a mammal in need thereof), particularly wherein the mammal is a human (such as a male or female human), by oral administration olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day.

In an another particular embodiment, the invention provides a method for the treatment of type II SMA, comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of type II SMA, comprising administering to a mammal (in particular a mammal in need thereof), particularly wherein the mammal is a human, such as a male or female human, particularly wherein the mammal is a human (such as a male or female human), by oral administration Olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day.

In an another particular embodiment, the invention provides a method for the treatment of type III SMA, comprising administering to a mammal (in particular a mammal in need thereof), particularly wherein the mammal is a human (such as a male or female human), olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day. More particularly, the invention provides a method for the treatment of type III SMA, comprising administering to a mammal (in particular a mammal in need thereof), particularly wherein the mammal is a human (such as a male or female human), by oral administration Olesoxime at more than 15 mg/kg per day, in particular at 15 mg/kg per day to 40 mg/kg per day, more particularly at 15 mg/kg per day to 30 mg/kg per day, even more particularly at 20 mg/kg per day to 30 mg/kg per day, most particularly at 20 mg/kg per day.

In another embodiment, the invention provides a method for the treatment of spinal muscular atrophy (SMA), in a mammal (in particular a mammal in need thereof), particularly wherein the mammal is a human (such as a male or female human), which comprises administering 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering 20 mg per kilogram of body weight of olesoxime per day.

In another embodiment, the invention provides a method for the treatment of spinal muscular atrophy (SMA), in a mammal (in particular a mammal in need thereof), particularly wherein the mammal is a human (such as a male or female human), which comprises administering orally 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering orally 20 mg per kilogram of body weight of olesoxime per day.

In another embodiment, the invention provides a method for the treatment of spinal muscular atrophy (SMA), in a mammal (in particular a mammal in need thereof), particularly wherein the mammal is a human (such as a male or female human), which comprises administering a dose of 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering a dose of 20 mg per kilogram of body weight of olesoxime per day.

In another embodiment, the invention provides a method for the treatment of spinal muscular atrophy (SMA), in a mammal (in particular a mammal in need thereof), particularly wherein the mammal is a human (such as a male or female human), which comprises administering orally a dose of 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering orally a dose of 20 mg per kilogram of body weight of olesoxime per day.

In another embodiment, the invention provides a method for the treatment of spinal muscular atrophy (SMA), in a mammal (in particular a mammal in need thereof), particularly wherein the mammal is a human (such as a male or female human), which comprises administering a dosage of 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering a dose of 20 mg per kilogram of body weight of olesoxime per day.

In another embodiment, the invention provides a method for the treatment of spinal muscular atrophy (SMA), in a mammal (in particular a mammal in need thereof), particularly wherein the mammal is a human (such as a male or female human), which comprises administering orally a dosage of 20 mg per kilogram of body weight of olesoxime. Particularly, the invention provides a method for the treatment of spinal muscular atrophy (SMA), in a mammal (in particular a mammal in need thereof), particularly wherein the mammal is a human (such as a male or female human), which comprises administering orally a dose of 20 mg per kilogram of body weight of olesoxime per day.

In a further embodiment, the invention provides a method of treating spinal muscular atrophy (SMA), comprising administering to a mammal olesoxime at more than 15 mg/kg, in particular at 15 mg/kg to 40 mg/kg, more particularly at 15 mg/kg to 30 mg/kg, even more particularly at 20 mg/kg to 30 mg/kg, most particularly at 20 mg/kg.

In another embodiment, the invention provides a pharmaceutical composition for use in the treatment of SMA (more particularly type II or/and type III SMA), which comprises more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime being administered, in particular orally administered once a day.

In another embodiment, the invention provides a pharmaceutical composition for use in the treatment of SMA (more particularly type II or/and type III SMA), which comprises 20 mg per kilogram of body weight to 30 mg per kilogram of body weight of olesoxime being administered, in particular orally administered once a day.

In another embodiment, the invention provides a pharmaceutical composition for use in the treatment of SMA (more particularly type II or/and type III SMA), which comprises 20 mg per kilogram of body weight of olesoxime being administered, in particular orally administered once a day.

The pharmaceutical composition can be used to treat spinal muscular atrophy (SMA), in particular type II SMA and type III SMA, in a mammal, especially a human (i. e., a male or female human).

In yet another embodiment, the invention provides a pharmaceutical composition for use in the treatment of SMA (more particularly type II or/and type III SMA), which comprises more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime, being administered, in particular orally administered once a day, wherein the composition comprises olesoxime and an oil chosen from sesame oil, olive oil, soya oil, cotton oil, or a mixture of medium-chain triglycerides (ESTASAN®, MYGLIOL®) or a mixture of the oils thereof.

In another embodiment, the invention provides a pharmaceutical composition for use in the treatment of SMA (more particularly type II or/and type III SMA), which comprises more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime being administered, in particular orally administered once a day, wherein the composition comprises olesoxime and an oil chosen from sesame oil, olive oil, soya oil, or a mixture of the oils thereof.

In yet another embodiment, the invention provides a pharmaceutical composition for use in the treatment of SMA (more particularly type II or/and type III SMA), which comprises more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime, in being administered, in particular orally administered once a day, wherein the composition comprises olesoxime and an oil chosen from sesame oil, olive oil or soya oil.

In yet another embodiment, the invention provides a pharmaceutical composition for use in the treatment of SMA (more particularly type II or/and type III SMA), which comprises more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime, being administered, in particular orally administered once a day, wherein the composition comprises olesoxime and sesame oil.

According to the invention, the compound can be present in the pharmaceutical composition as herein described in a quantity ranging from 10 to 200 mg/ml of olesoxime in solution, particularly from 25 to 150 mg/ml, or in a quantity ranging from 30 to 500 mg/ml of olesoxime in suspension, particularly from 50 to 400 mg/ml, more particularly at 100 mg/mL.

In another embodiment, the invention provides the use of more than 15 mg per kilogram of body weight of olesoxime, particularly 15 mg per kilogram of body weight to 40 mg per kilogram of body weight of olesoxime, more particularly 15 mg per kilogram of body weight to 30 mg per kilogram of body weight of olesoxime, even more particularly at 20 mg per kilogram of body weight to 30 mg per kilogram of body weight of olesoxime, most particularly 20 mg per kilogram of body weight of olesoxime for the treatment of SMA, in particular type II SMA or/and type III SMA.

In another embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day, of olesoxime for the treatment of SMA, in particular type II SMA or/and type III SMA.

In a particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the treatment of SMA in mammal (in particular a mammal in need thereof), particularly wherein the mammal is a human (such as a male or female human).

In a particular embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day, of olesoxime for the treatment of SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the treatment of type II SMA or/and type III SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the treatment of type II SMA or type III SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the treatment of type II SMA and type III SMA in mammal (in particular a mammal in need thereof), particularly wherein the mammal is a human (such as a male or female human).

In a more particular embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day, of olesoxime for the treatment of type II SMA and type III SMA in mammal (in particular a mammal in need thereof), particularly wherein the mammal is a human (such as a male or female human).

In another embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the preparation of medicaments for the treatment of SMA, in particular type II SMA or/and type III SMA.

In another embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day of olesoxime for the preparation of medicaments for the treatment of SMA, in particular type II SMA or/and type III SMA.

In a particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight of olesoxime for the preparation of medicaments for the treatment of SMA in mammal (in particular a mammal in need thereof), particularly wherein the mammal is a human (such as a male or female human).

In a particular embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day, of olesoxime for the preparation of medicaments for the treatment of SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the preparation of medicaments for the treatment of type II SMA or/and type III SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the preparation of medicaments for the treatment of type II SMA or type III SMA in mammal (in particular a mammal in need thereof), particularly wherein the mammal is a human (such as a male or female human).

In a more particular embodiment, the invention provides the use of more than 15 mg per kilogram of body weight, particularly 15 mg per kilogram of body weight to 40 mg per kilogram of body weight, more particularly 15 mg per kilogram of body weight to 30 mg per kilogram of body weight, even more particularly at 20 mg per kilogram of body weight to 30 mg per kilogram of body weight, most particularly 20 mg per kilogram of body weight, of olesoxime for the preparation of medicaments for the treatment of type II SMA and type III SMA in mammal (in particular a mammal in need thereof), particularly wherein the mammal is a human (such as a male or female human).

In a more particular embodiment, the invention provides the use of more than 15 mg per kilogram of body weight per day, particularly 15 mg per kilogram of body weight to 40 mg per kilogram of body weight per day, more particularly 15 mg per kilogram of body weight to 30 mg per kilogram of body weight per day, even more particularly at 20 mg per kilogram of body weight to 30 mg per kilogram of body weight per day, most particularly 20 mg per kilogram of body weight per day, of olesoxime for the preparation of medicaments for the treatment of type II SMA and type III SMA in mammal (in particular a mammal in need thereof), particularly wherein the mammal is a human (such as a male or female human).

The mammal according to the invention in particular is a human, more particularly a male or female human. The human can be of any race (e. g., Caucasian or Oriental).

In particular embodiments, the methods, the uses, pharmaceutical compositions in accordance with the present invention, olesoxime is administered once a day, more particularly with food, most particularly with the evening meal.

The following example is intended merely to illustrate the practice of the present invention and is not provided by way of limitation.

### EXAMPLE 1

A randomized, double-blind, placebo-controlled, Phase 2 study (NCT01302600) was performed in 22 secondary care centres in Belgium, France, Germany, Italy, Netherlands, Poland and the UK. Safety and efficacy of olesoxime (TRO19622/RG6083) were assessed in patients aged 3-25 years with type 2 or non-ambulatory type 3 SMA confirmed by weakness and hypotonia, genetic diagnosis, Motor Function Measure (MFM) domains 1 and 2 (D1+D2) relative score (percentage of the maximum sum)≥15% and Hammersmith Functional Motor Scale (HFMS) score at baseline≥3 (non-ambulant≤38). Patients were randomized centrally by a computer-generated sequence to receive olesoxime 10 mg/kg oral liquid suspension or placebo (2:1 ratio) for 104 weeks. 198 patients were recruited between November 2010 and September 2011, and 165 were randomized to olesoxime (n=108) or placebo (n=57).

MFM is a validated quantitative scale allowing measurement of the functional motor abilities of an individual affected by a neuromuscular disease, regardless of the diagnosis and the extent of motor deficiencies (Vuillerot et al. Arch Phys Med Rehabil 2013; 94(8): 1555-61). The MFM contains 32 assessments of motor function and is administered by a trained rater, including clinicians and physiotherapists (Vuillerot et al. Arch Phys Med Rehabil 2013; 94(8): 1555-61). Factor analysis identified three dimensions, assessing standing and transfers (D1, 13 items), axial and proximal function (D2, 12 items), and distal motor function (D3, 7 items). Each item is scored on a 4-point (0-3) Likert type scale, where each option contains a clear indication of the level to which the patient has achieved the functional task and higher scores reflect better functional ability. The scoring 4-point Likert scale is based on the subject's maximal abilities without assistance (Vuillerot et al. Arch Phys Med Rehabil 2013; 94(8): 1555-61). The point scale is as follows:
0, cannot initiate the task or maintain the starting position;
1, performs the task partially;
2, performs the task incompletely or imperfectly (with compensatory/uncontrolled movements or slowness);
3, performs the task fully and “normally.”

A total score, in addition to the three dimensions, can be calculated, with all scores expressed as a percentage of the maximum possible sum of the item scores. This applies to all versions of the MFM.
The D1 dimension assesses standing, ambulation, and transfers between positions (e.g., from supine to sitting).
The D2 dimension assesses tasks from supine or seated positions, and tasks related to axial and proximal function (e.g., ability to raise their arms and touch their head).
The D3 dimension assesses distal function, including several assessments of fine motor skills, such as picking up coins.

Evidence of strong measurement properties (validity, reliability, and ability to detect change) for the MFM D1+D2 score was identified using data from the herewith referred clinical trial.

Of 108 patients allocated to olesoxime, five patients were excluded from the FAS due to the absence of post-baseline assessments (Vuillerot C, et al., Arch Phys Med Rehabil 2013; 94(8): 1555). Major protocol violations occurred in 30 patients (olesoxime n=19 [18%], placebo n=11 [19%]), including 17 patients who performed the incorrect version of the MFM. Other major protocol violations included deviation from the inclusion screening criteria (use of forbidden medication, spinal rod or fixation for scoliosis within six months of enrolment), abnormal liver enzymes (ALT or AST>3×upper limits of normal), treatment compliance≤50% for at least two visits and delayed visit dates. The full analysis set (FAS) comprised 160 patients (olesoxime: n=103; placebo: n=57) (Table 1). All efficacy analyses reported here were based on the Full Analysis Set (FAS).

TABLE 1
Patient demographics (FAS)

| | | Olesoxime | | Placebo | | Total | |
|---|---|---|---|---|---|---|---|
| | | n = 103 | | n = 57 | | N = 160 | |
| Sex, n (%) | Male | 55 | (53.4) | 25 | (43.9) | 80 | (50.0) |
| | Female | 45 | (46.6) | 32 | (56.1) | 80 | (50.0) |
| Age at | Mean ± SD | 9.12 ± 5.48 | | 11.2 ± 5.98 | | 9.88 ± 5.74 | |
| baseline | Median | 7 | | 11 | | 8 | |
| (years) | Range | 3-25 | | 3-27 | | 3-27 | |
| Age | <6 years | 35 | (34.0%) | 14 | (24.6%) | 49 | (30.6%) |
| categories, | ≥6 years | 68 | (66.0%) | 43 | (75.4%) | 111 | (69.4%) |
| n (%) | | | | | | | |
| SMA type, | Type II | 74 | (71.8%) | 39 | (68.4%) | 113 | (70.6%) |
| n (%) | Type III | 29 | (28.2%) | 18 | (31.6%) | 47 | (29.4%) |

The pharmacokinetics (PK) and efficacy data from the herewith referred clinical trial study were used to investigate the relationship between olesoxime pharmacokinetic exposure (i.e., average trough concentration Caverage) and the primary efficacy outcome (i.e.: MFM D1+D2 score). A longitudinal kinetic-pharmacodynamic (KPD) approach was applied [Jacqmin et al., 2007]. In the placebo group the progression of the disease over time was best described by a linear function (slope of −0.0201 unit/week with relative standard error of 27%). The exposure-response relationship was also best described by a linear function (slope of 0.291 unit/ng/mL with a relative standard error of 36%) indicating that the effect of Olesozime did not reach a plateau at the dose of 10 mg and that higher doses should lead to an increase in efficacy. (Data on file).

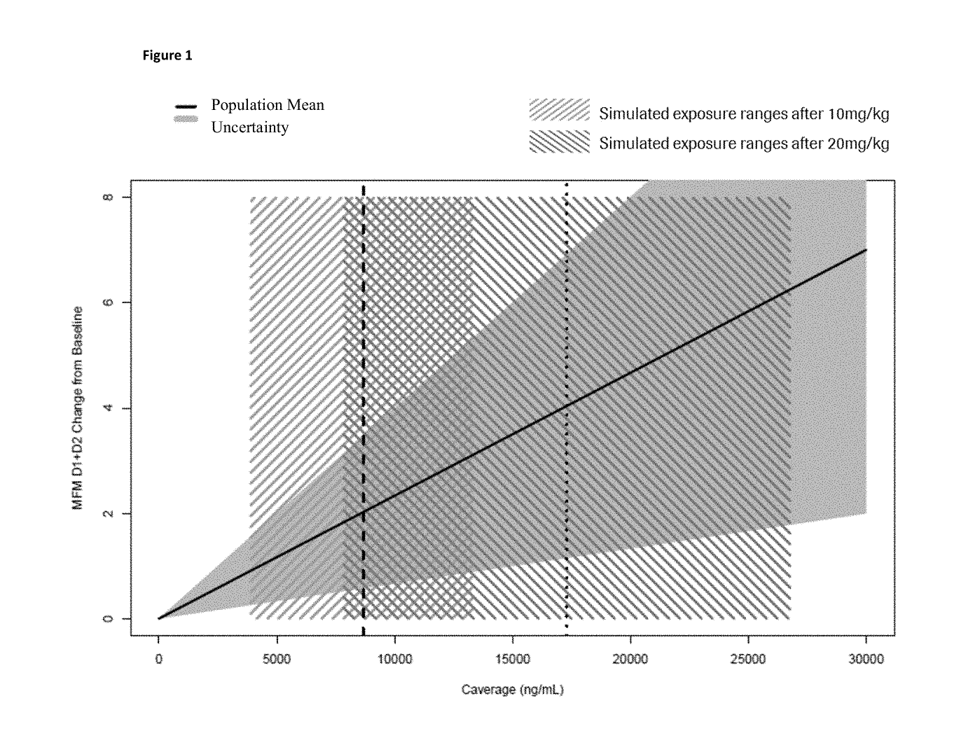

Simulations were conducted by using this exposure-response function and its associated uncertainty. By assuming proportionality between olexosime dose and PK exposure a dose of 20 mg/kg is expected to provide a clinically response over baseline at 2 years of four point improvement in the primary endpoint MFM D1+D2 (FIG. 1) which is two-point improvement compare to 10 mg/kg.

### EXAMPLE 2

Deletion of the smn gene is lethal in mice (early embryonic lethality). Mutant mice with deletion of smn exon7 restricted to neurons (NSE-Cre; SMNF7/F7, referred to here as SMA transgenic mice) are viable and display a phenotype similar to that described in human patients suffering from the severe types of spinal muscular atrophy (Frugier T., Tiziano F D et al. Hum Mol Genet. 2000, 9, 849-858.)

A pre-clinical study has been carried out on transgenic mouse model of SMA in the severe modelSMN^{F7/F7}; NSE-Cre with Olesoxime to test for signs of possible efficacy to treats SMA patients. The experimental groups were spitted as shown in table 2.

TABLE 2
Distribution of Mice according to genotype, sex and treatment

| | SMN^{F7/F7}; | | |
|---|---|---|---|
| | NSE-Cre | SMN^{F7-} | |
| | Effected Mice | Control Mice | |
| Group | Sex Ratio (M/F) | Sex Ratio M/F | |
| Vehicle | 40 (17/23) | 15 | (10/5) |
| 10 mg/Kg | 45 (24/21) | 15 | (9/6) |
| 30 mg/ kg | 43 (23/20) | 15 | (7/8) |

Endpoint scores included survival (daily observation), body weight (animals were weighed 3 times/week).

### Preparation of the Treatment:

Olesoxime (powder) was mixed with Cremophore EL (SIGMA C5135), absolute ethanol (CARLO ERBA RPE 414571) and DMSO (ALLDRICH 27685-5) (5/5/10, respective % of the final volume). After complete dissolution, PBS (phosphate buffered saline) was added (85% of the final volume). Solutions were prepared in volumes sufficient for 3 days; vehicle placebo consisted in the same mixture of excipients without the active principle. Daily treatment (subcutaneous injections, 10 ml/kg) started from P21 to death. Effected mice were killed by cervical dislocation when they became unable to feed, according to ethical considerations or around 45 days for control mice. Experiments were performed blind.

### Methods and Tools:

“log Rank”, also called “Mantel-Haenszel” was the statistic test being used to compare the two survival curves. It is a type “Khi2” test that was already available in the library “survival” from R. software (http://lib.stat.cmu.edu/R/CRAN).

The test was applied simultaneously on the 3 groups of mice and for all five stages.

All the mice which died before day 26 were removed from the study. There were 8 in total. The final results did not seem to be affected by this.

### Results:

Olesoxime activity was evaluated in this model. Transgenic mice were treated daily by subcutaneous injection of olesoxime at 10 or 30 mg/kg or vehicle, starting at 21 days of age until the end of their life. Olesoxime induced a significant increase in life span in mice treated with 30 mg/kg (log rank test, p<0.05; Error! Reference source not found. Only 15% of vehicle-treated animals survived for longer than 40 days whereas 25% and 45% of the olesoxime-treated animals were still alive after 40 days when treated daily with 10 or 30 mg/kg, respectively. There was no difference in body weight in any of the groups.

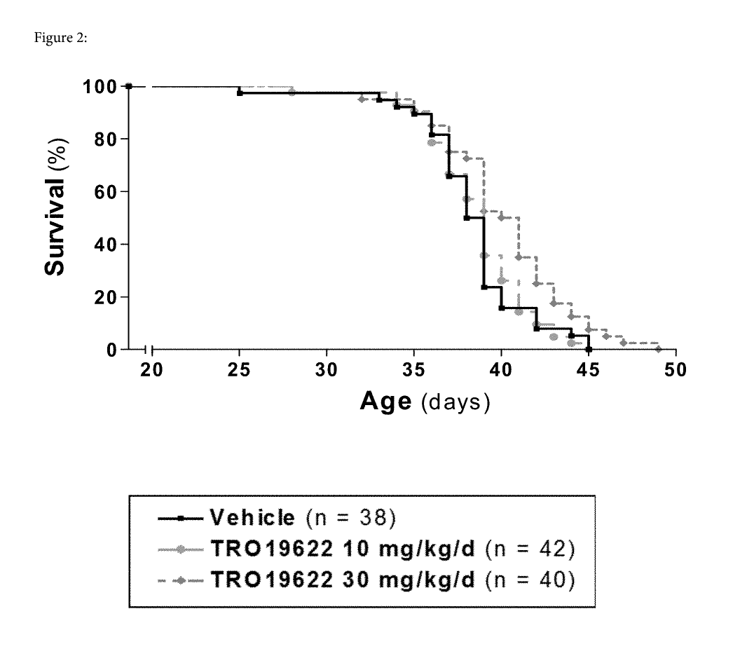

The difference of the group is significant (with a P value of less 0.05) for stage 5, wherein the mice dead before the 26^{th} days have been removed, as illustrated by FIG. 2.

TABLE 3
Collected Mice data: Group A is Placebo group;
Group B is the 10 mg/Kg; Group C is the 30 mg/Kg
New treatment of SMA

| Souris | Group | Sex | stage5 |
|---|---|---|---|
| 4306 | A | F | 25 |
| 4310 | A | F | 39 |
| 4352 | A | F | 39 |
| 4353 | A | F | 35 |
| 4359 | A | F | 39 |
| 4360 | A | F | 38 |
| 4585 | A | F | 45 |
| 4386 | A | F | 44 |
| 4588 | A | F | 45 |
| 4698 | A | F | 38 |
| 4789 | A | F | 37 |
| 4948 | A | F | 39 |
| 5887 | A | F | 33 |
| 5891 | A | F | 36 |
| 5894 | A | F | 36 |
| 5896 | A | F | 37 |
| 5899 | A | F | 40 |
| 6149 | A | F | 38 |
| 6153 | A | F | 42 |
| 6202 | A | F | 37 |
| 6280 | A | F | 42 |
| 6281 | A | F | 40 |
| 4307 | A | M | 39 |
| 4348 | A | M | 39 |
| 4349 | A | M | 39 |
| 4502 | A | M | 24 |
| 4786 | A | M | 22 |
| 4788 | A | M | 38 |
| 4824 | A | M | 34 |
| 4946 | A | M | 39 |
| 5883 | A | M | 38 |
| 5866 | A | M | 38 |
| 5889 | A | M | 37 |
| 5895 | A | M | 37 |
| 5987 | A | M | 38 |
| 6105 | A | M | 39 |
| 6106 | A | M | 40 |
| 6112 | A | M | 39 |
| 6150 | A | M | 42 |
| 6203 | A | M | 37 |
| 4428 | B | F | 34 |
| 4528 | B | F | 37 |
| 4540 | B | F | 36 |
| 4541 | B | F | 36 |
| 4632 | B | F | 36 |
| 4633 | B | F | 40 |
| 4534 | B | F | 26 |
| 4617 | B | F | 37 |
| 4366 | B | F | 39 |
| 4367 | B | F | 39 |
| 4984 | B | F | 43 |
| 5903 | B | F | 39 |
| 5994 | B | F | 39 |
| 6118 | B | F | 45 |
| 6129 | B | F | 39 |
| 6136 | B | F | 38 |
| 6139 | B | F | 37 |
| 6142 | B | F | 42 |
| 6256 | B | F | 34 |
| 6424 | B | F | 25 |
| 6453 | B | F | 38 |
| 4470 | B | M | 40 |
| 4537 | B | M | 40 |
| 4538 | B | M | 39 |
| 4539 | B | M | 39 |
| 4772 | B | M | 36 |
| 4815 | B | M | 38 |
| 4816 | B | M | 37 |
| 4980 | B | M | 40 |
| 4981 | B | M | 37 |
| 5905 | B | M | 36 |
| 5914 | B | M | 38 |
| 5916 | B | M | 25 |
| 5993 | B | M | 39 |
| 6116 | B | M | 39 |
| 6130 | B | M | 35 |
| 6141 | B | M | 44 |
| 6249 | B | M | 41 |
| 6250 | B | M | 41 |
| 6253 | B | M | 41 |
| 6414 | B | M | 43 |
| 6418 | B | M | 23 |
| 6449 | B | M | 42 |
| 6450 | B | M | 41 |
| 6451 | B | M | 41 |
| 3235 | C | F | 39 |
| 4344 | C | F | 35 |
| 4363 | C | F | 45 |
| 4375 | C | F | 41 |
| 4377 | C | F | 49 |
| 4662 | C | F | 43 |
| 4798 | C | F | 37 |
| 4832 | C | F | 39 |
| 4972 | C | F | 47 |
| 5982 | C | F | 40 |
| 6055 | C | F | 35 |
| 6064 | C | F | 42 |
| 6207 | C | F | 41 |
| 6233 | C | F | 38 |
| 6235 | C | F | 39 |
| 6273 | C | F | 41 |
| 6397 | C | F | 42 |
| 6438 | C | F | 37 |
| 6442 | C | F | 41 |
| 6459 | C | F | 44 |
| 6526 | C | F | 39 |
| 4312 | C | M | 43 |
| 4316 | C | M | 39 |
| 4340 | C | M | 36 |
| 4684 | C | M | 42 |
| 4685 | C | M | 37 |
| 4686 | C | M | 46 |
| 4687 | C | M | 39 |
| 4688 | C | M | 42 |
| 4691 | C | M | 44 |
| 4828 | C | M | 28 |
| 4830 | C | M | 36 |
| 4859 | C | M | 32 |
| 5946 | C | M | 39 |
| 5963 | C | M | 41 |
| 5970 | C | M | 39 |
| 6062 | C | M | 23 |
| 6121 | C | M | 45 |
| 6122 | C | M | 41 |
| 6209 | C | M | 37 |
| 6229 | C | M | 43 |
| 6277 | C | M | 39 |
| 6443 | C | M | 22 |

### CONCLUSION

The main result of this study in 128 mice shows that (SMNF7/F7; NSE-Cre) mice treated daily with 30 mg/kg s.c. olesoxime from day 21 after birth to death have a longer life span than mice treated with the placebo. Only 15% of the vehicle treated mice survived more than 40 days whereas 26% and 50% were still alive at 40 days in the groups treated with 10 and 30 mg/kg respectively. The difference of the group is significant (with a P value of less 0.05) for survival, wherein the mice dead before the 26^{th} days have been removed, as illustrated by FIG. 2.

1.-7. (canceled)

8. A method for the treatment of SMA, comprising orally administering olesoxime to a human at a dose of 15 mg/kg to 40 mg/kg per day.

9. The method of claim 8, wherein the dose is 20 mg/kg to 30 mg/kg per day.

10. The method of claim 8, wherein the dose is 15 mg/kg to 20 mg/kg per day.

11. The method of claim 8, wherein SMA is of type II SMA or type III SMA.

12. A method for the treatment of SMA, in a human which comprises administering orally 20 mg per kilogram of body weight of olesoxime per day.

13. (canceled)

14. (canceled)

15. The method of claim 12, wherein SMA is of type II SMA or type III SMA.

16.-25. (canceled)

26. The method of claim 9, wherein SMA is of type II SMA or type III SMA.

27. The method of claim 10, wherein SMA is of type II SMA or type III SMA.

28. The method of claim 8, wherein the dose of olesoxime is administered once daily.

29. The method of claim 9, wherein the dose of olesoxime is administered once daily.

30. The method of claim 10, wherein the dose of olesoxime is administered once daily.

31. The method of claim 11, wherein the dose of olesoxime is administered once daily.

32. The method of claim 12, wherein the dose of olesoxime is administered once daily.

33. The method of claim 15, wherein the dose of olesoxime is administered once daily.
